(19) United States
(12) Patent Application Publication
(10) Pub. No.: US 2007/0060887 A1
(43) Pub. Date: Mar. 15, 2007

(54) Ophthalmic injector

(76) Inventors: David A. Marsh, Fort Worth, TX (US); Theron R. Rodstrom, Cranfills Gap, TX (US); Alan L. Weiner, Arlington, TX (US)

Correspondence Address: ALCON, IP LEGAL, TB4-8, 6201 SOUTH FREEWAY, FORT WORTH, TX, 76134, US

(21) Appl. No.: 11/486,870

(22) Filed: Jul. 14, 2006

Related U.S. Application Data
(60) Provisional application No. 60/710,046, filed on Aug. 22, 2005.

(51) Int. Cl. A61F 7/12

(52) U.S. Cl. 604/113

(57) ABSTRACT

An ophthalmic injector having a nose cone with a heating chamber, a heater assembly, and a needle that provide improved drug delivery.

This application claims the priority of U.S. Provisional Application No. 60/710,046 filed Aug. 22, 2005.

### FIELD OF THE INVENTION

The present invention generally pertains to ophthalmic drug delivery and more particularly to posterior segment ophthalmic drug delivery.

### DESCRIPTION OF THE RELATED ART

Current intravitreal drug delivery devices deliver drugs to the vitreous through the pars plana region of the eye, which is devoid of retinal tissue, to avoid complications such as retinal detachment. Examples include the VITRASERT® implant and the RETISERT® implant available from Bausch & Lomb, both of which require a relatively large pars plana incision (e.g. 1-5 mm) for implantation. The OCUSERT device, which has been used to deliver pilocarpine for the treatment of glaucoma, employs a pars plana injection of biodegradable or bioerodible pellets through a 22 gauge needle, which is not “self-sealing” procedure.

U.S. Pat. No. 4,030,499 to Bucalo discloses a syringe containing an implant in solid condition. The syringe includes a heating coil disposed on the exterior of or on the interior of the syringe barrel and the associated needle for converting the solid implant into a liquid prior to injection into the body tissue. However, such a coil design may result in increased manufacturing cost and potential reliability issues. U.S. Pat. No. 6,488,659 to Rosenman discloses a catheter with heat transferring fluid passageways for injecting a thermally sensitive gelation material to remote sites within a patient's body. The thermally sensitive gelation material is delivered to the catheter via a syringe. The catheter maintains the thermally sensitive gelation material in a liquid state until it is delivered to the target issue. However, catheterized drug delivery is not suitable for ophthalmic applications.

Several diseases and conditions of the posterior segment of the eye continue to threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples. Therefore, a need continues to exist for improved posterior segment ophthalmic drug delivery that does not suffer from the above-described limitations of current devices.

### SUMMARY OF THE INVENTION

In one aspect, the present invention is an ophthalmic injector including a nose cone, a dosage form, a needle, a plunger assembly, and a heater assembly. The nose cone has a heating chamber. The dosage form is in a solid state and is disposed in the heating chamber. The needle is fluidly coupled to the heating chamber, and the plunger assembly has a rod at least partially disposed in the heating chamber. The heater assembly is disposed on the nose cone. The heater assembly heats the nose cone and the heating chamber to cause the dosage form to change from a solid state to a liquid state, and the nose cone transfers sufficient heat to the needle to prevent the dosage form in the liquid state from re-solidifying within the needle.

Another aspect of the present invention is an ophthalmic injector including a heating chamber, a dosage form, a needle, a plunger assembly, and a heater assembly. The dosage form is in a solid state and is disposed in the heating chamber. The needle is fluidly coupled to the heating chamber and has a length and a diameter enabling insertion into the vitreous at points other than the pars plana. The plunger assembly has a rod at least partially disposed in the heating chamber. The heater assembly is for heating the heating chamber to cause the dosage form to change from a solid state to a liquid state to enable the rod to inject the dosage form through the needle.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a plan view of an ophthalmic injector according to a preferred embodiment of the present invention;

FIG. 2 is a side, sectional view of the injector of FIG. 1 taken along line 2-2; and

FIG. 3 is an exploded, perspective view of the injector of FIG. 1.

### DETAILED DESCRIPTION OF THE PREFERRED EMBODIMENTS

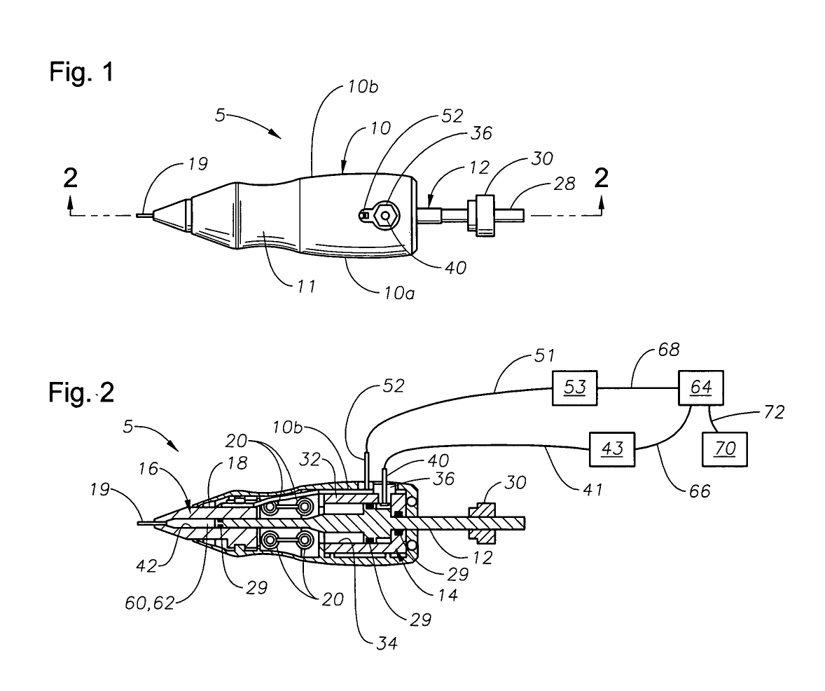

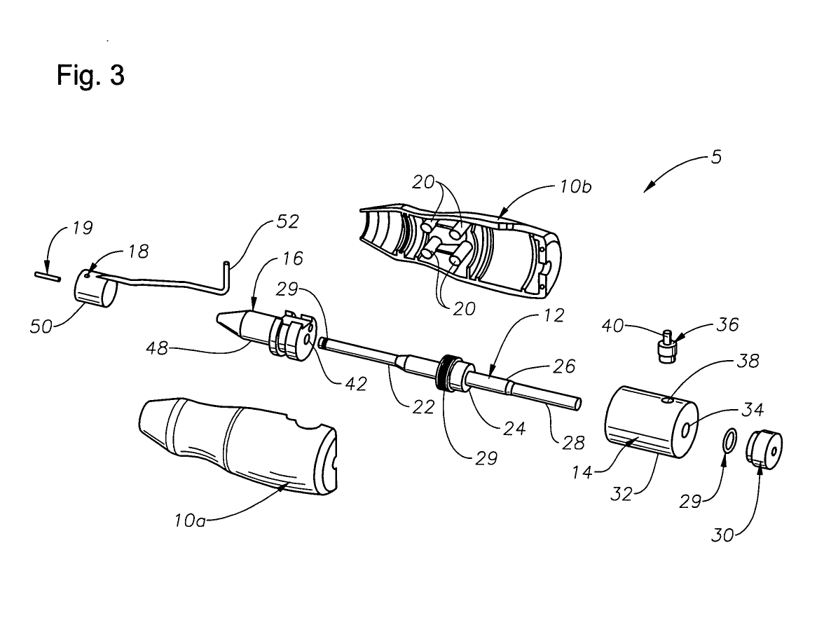

The preferred embodiments of the present invention and their advantages are best understood by referring to FIGS. 1-3 of the drawings, like numerals being used for like and corresponding parts of the various drawings.

Ophthalmic injector 5 generally includes a housing 10, a plunger assembly 12, an actuation assembly 14, a nose cone 16, a heater assembly 18, a needle 19, and a dosage form 60. Housing 10 includes a left housing 10a and a right housing 10b that are removably and frictionally coupled via pin mounts 20 and pins (not shown). Housing 10 preferably includes a region 11 for grasping ophthalmic injector 5. Plunger assembly 12 preferably includes a distal rod 22, a base 24, and a proximal rod 26. Rod 26 has a threaded end 28 for rotationally coupling with an adjustment nut 30. Plunger assembly 12 also includes o-rings 29 for fluidly sealing to various surfaces of ophthalmic injector 5. Actuation assembly 14 is preferably a cylinder 32 having a bore 34 for receiving plunger assembly 12 and for holding a volume of pressurized fluid. The pressurized fluid is preferably air. Cylinder 32 preferably has a fitting 36 for disposing within an opening 38. Fitting 36 has a lumen 40 in fluid communication with bore 34. Lumen 40 is fluidly coupled to tubing 41. Tubing 41 is for fluidly coupling with a source of pressurized fluid 43 external to ophthalmic injector 5. Nose cone 16 preferably includes a heating chamber 42 and a heater assembly mount 48. Nose cone 16 is preferably made from a material having a high thermal conductivity and is most preferably aluminum. Heater assembly 18 has a body 50 having a geometry for mating with heater assembly mount 48. Heater assembly 18 also has power leads 52 that are electrically coupled to an interface 51. Interface 51 is for electrically coupling to a power source 53 external to ophthalmic injector 5. Needle 19 is a self-sealing needle that is in fluid communication with heating chamber 42. Needle 19 is preferably a 23-25 gage, stainless steel needle having a length of about 0.5 to about 5 mm.

A dosage form 60 including a drug 62 is disposed in heating chamber 42 in a sterilized manner during the manufacture of ophthalmic injector 5. Dosage form 60 is a low melting point dosage form that is capable of delivery to the eye in a liquid state, solidifying at the target site, and delivering a controlled or sustained release amount of drug 62 to the target site. Dosage form 60 preferably has a melting point between about 40° C. to about 80° C. and is preferably biodegradable or bioerodible upon solidifying in vivo. Dosage form 60 is preferably heated, disposed within heating chamber 42 in a liquid state, and then solidified. Heating chamber 42 preferably holds about 1 to about 100 μL of dosage form 60. Of course, the desired volume of dosage form 60 may vary for dosage forms with different drugs 62. Drug 62 may be any ophthalmically acceptable drug. Drug 62 is preferably an ophthalmically acceptable drug for the treatment or prevention of a disease or condition of the posterior segment of the eye, including age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies. Dosage form 60 may also include other ophthalmically acceptable excipients, including excipients to alter its melting point.

Once dosage form 60 is disposed within heating chamber 42, left housing 10a and right housing 10b may be secured via pins and pin mounts 20. Adjustment nut 30 is then moved to the proper position along threaded end 28 of proximal rod 26 to adjust the throw of plunger assembly 12 for the proper volume of dosage form 60. Adjustment nut 30 is then preferably locked into place, preventing subsequent adjustment. Ophthalmic injector 5 may then be placed in its final packaging and sterilized.

Source of pressurized fluid 43 and power source 53 are preferably electrically coupled to a computer or microprocessor 64 via interfaces 66 and 68, respectively. A controller 70 is also preferably electrically coupled to microprocessor 64 via an interface 72. Controller 70 is preferably a foot controller.

A preferred method of using ophthalmic injector 5 to deliver a drug to the eye will now be described in greater detail. Ophthalmic injector 5 is particularly useful for drug delivery to the posterior segment of the eye. Because of the short length and self-sealing nature of needle 19, ophthalmic injector 5 can be inserted into the vitreous at locations other than the pars plana with a very low likelihood of disturbing the bulk of retinal tissue, even if needle 19 is inserted through the retina. The ability of ophthalmic injector 5 to inject a dosage form anywhere in the posterior segment maximizes drug concentration at the specific target tissue and decreases the potential for toxic side effects. In addition, ophthalmic injector 5 can be used to inject a dosage form to other portions of posterior segment or the anterior segment of the eye.

Prior to drug delivery, a nurse fluidly couples tubing 41 to source of pressurized fluid 43 and electrically couples interface 51 to power source 53. Within the sterile field, the physician activates power source 53 via controller 70. Heater assembly 18 heats nose cone 16, and thus heating chamber 42, so that dosage form 60 quickly transforms from a solid to a liquid state. Microprocessor 64 preferably customizes the amount of power and the heating time for a specific dosage form 60. The physician grasps ophthalmic injector 5 via region 11 and begins the delivery procedure on an anesthetized patient. Needle 19 is inserted into the vitreous of the eye at a portion of the posterior segment proximate the target tissue. Acutation assembly 14 is actuated via controller 70 to move distal rod 22 into heating chamber 42. Microprocessor 64 preferably controls the actuation pressure and time for a specific dosage form 60. Liquefied dosage form 60 flows from heating chamber 42, through needle 19, and into the target tissue. Once at the target tissue, dosage form 60 solidifies on the target tissue and begins controlled or sustained release delivery of drug 62. The physician removes needle 19 from the vitreous, and the opening caused by needle 19 self seals. Tubing 41 and interface 51 are uncoupled from power source 43 and source of pressurized fluid 53, respectively. Ophthalmic injector 5 is then preferably disposed of in a sharps collector.

Microprocessor 64 controls power source 53 so that heater assembly 18 heats dosage form 60 to a temperature where it remains in liquid form during its passage through needle 19 and until it reaches the target tissue but below a temperature where it could potentially damage or irritate the target tissue. Passive heat transfer between nose cone 16 and needle 19 preferably facilitates this process and eliminates the need to actively head needle 19 via an electric coil or other heating apparatus.

From the above, it may be appreciated that the present invention provides improved devices and methods for safe, effective, rate-controlled, localized delivery of a variety of drugs to the eye, and particularly to the posterior segment of the eye. The present invention is illustrated herein by example, and various modifications may be made by a person of ordinary skill in the art. For example, while the present invention is described above as using a plunger assembly 12 having a pneumatic actuation assembly 14, various other mechanical or electro-mechanical plunger and actuation assemblies may be utilized, such as a spring-actuated plunger assembly, an electrically powered linear actuator with plunger, or an electrically powered stepper motor with plunger. As another example, injector 5 may be used to deliver drugs to target tissues within the body other than the eye.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. An ophthalmic injector, comprising:
a nose cone having a heating chamber;
a dosage form in a solid state disposed in said heating chamber;
a needle fluidly coupled to said heating chamber;
a plunger assembly having a rod at least partially disposed in said heating chamber; and
and a heater assembly disposed on said nose cone;
whereby said heater assembly heats said nose cone and said heating chamber to cause said dosage form to change from a solid state to a liquid state, and said nose cone transfers sufficient heat to said needle to prevent said dosage form in said liquid state from re-solidifying within said needle.

2. The ophthalmic injector of claim 1 wherein said heater assembly does not actively heat said needle.

3. An injector for an eye, said eye having a pars plana, a vitreous, and a retina, comprising:
a heating chamber;
a dosage form in a solid state disposed in said heating chamber;
a needle fluidly coupled to said heating chamber, said needle having a length and a diameter enabling insertion into said vitreous at points other than said pars plana;
a plunger assembly having a rod at least partially disposed in said heating chamber; and
a heater assembly for heating said heating chamber to cause said dosage form to change from a solid state to a liquid state to enable said rod to inject said dosage form through said needle.

4. The injector of claim 3 wherein said needle has a gauge of 23 or higher.

5. The injector of claim 4 wherein said needle has a gauge of 23 to 25.

6. The injector of claim 3 wherein said needle has a length of about 0.5 mm to about 5 mm.

7. The injector of claim 5 wherein said needle has a length of about 0.5 mm to about 5 mm.

8. The injector of claim 3 wherein said needle enables insertion through said retina into said vitreous.

9. The injector of claim 8 wherein said needle enables insertion through said retina into said vitreous without detachment of said retina.

10. The injector of claim 3 wherein said needle is self-sealing.
